(19) United States
(12) Patent Application Publication
(10) Pub. No.: US 2024/0307182 A1
(43) Pub. Date: Sep. 19, 2024

(54) FLAIL/PROLAPSE PREVENTING GUARD, METHODS OF USE AND KITS THEREOF

(71) Applicant: Valcare Medical, Inc., Wilmington, DE (US)

(72) Inventors: Andrea COLLI, Livorno (IT); Alessandro FIOCCO, Treviso (IT); Shuki PORATH, Haifa (IL); Nadav YELLIN, Aven Yehuda (IL); Yoav ROZEN, Binyamina (IL); Gal DEZYACHO, Herzelyia (IL); David MEERKIN, Jerusalem (IL)

(73) Assignee: Valcare Medical, Inc., Wilmington, DE (US)

(21) Appl. No.: 18/604,371

(22) Filed: Mar. 13, 2024

Related U.S. Application Data
(60) Provisional application No. 63/490,040, filed on Mar. 14, 2023.

(51) Int. Cl. A61F 2/24

(52) U.S. Cl. CPC A61F 2/2454; A61F 2/2445; A61F 2/2466; A61F 2230/0091

(57) ABSTRACT

The present disclosure relates to a guard member for preventing prolapse, flail, or billow of at least one leaflet of a heart valve, and a pathology associated therewith. Specifically, the guard member is adapted, sized and configured to operably couple to the heart valve annulus and prevent a leaflet of the heart valve from extending cephalad to the heart valve's annular plane.

### CROSS-REFERENCE TO RELATED APPLICATIONS

This application claims priority to U.S. Provisional Patent App. No. 63/490,040, filed Mar. 14, 2023, the entire contents of which are incorporated herein by reference.

### TECHNICAL FIELD

The present disclosure is directed to a guard member for preventing prolapse, flail, or billow of at least one leaflet of a heart valve, and a pathology associated therewith. Specifically, the guard member is adapted, sized and configured to operably couple to the heart valve annulus and prevent a leaflet of the heart valve from extending cephalad (in other words, upwards) to the heart valve's annular plane.

### BACKGROUND

Tricuspid, and/or mitral regurgitation (TR, MR) typically result from either intrinsic valve abnormalities or functional malcoaptation of structurally normal valves. For example, mitral valve prolapse is the most common cause of mitral regurgitation (MR) referred for surgery in the Western world. In addition, acute rheumatic carditis may also cause mitral valve prolapse. Likewise, TR caused by flail leaflets is most often posttraumatic, and can be caused by endocarditis or is a consequence of a myxomatously degenerated valve.

Echocardiography, especially two-dimensional transthoracic (TTE) and transesophageal echocardiography (TEE) are recognized as the preferred and more accurate diagnostic methods for detecting and diagnosing TR and/or MR resulting from prolapse and/or flail, and/or billow of a leaflet or portion thereof, and the differentiation of the etiology (e.g., among the prolapse and/or flail, and/or billow).

The most common treatments for mitral valve regurgitation rely on valve replacement or strengthening of the valve annulus by implanting a mechanical support ring or other structure. The latter is generally referred to as valve annuloplasty. Additional technique for mitral valve repair relies on suturing adjacent segments of the opposed valve leaflets together is referred to as the “bow-tie” or “edge-to-edge” technique. While all these techniques can be very effective, they usually rely on open heart surgery where the patient's chest is opened, typically via a sternotomy, and the patient placed on cardiopulmonary bypass. The need to both open the chest and place the patient on bypass is traumatic and has associated morbidity.

The proposed technology aims to address the condition.

### SUMMARY

In an exemplary implementation, provided herein is guard member for preventing prolapse, flail, or billow and a pathology associated therewith of at least one leaflet of a heart valve, the guard member adapted, sized and configured to operably couple to the heart valve annulus and prevent a first leaflet of the heart valve from extending cephalad to the heart valve's annular plane.

In another exemplary implementation, provided herein is Use of a first guard member configured to transition from an insertion configuration to a deployment configuration, the first guard member adapted sized and configured to be coupled to a heart valve annulus, operable to prevent a first leaflet of the heart valve leaflets from extending cephalad to the heart valve's annular plane, in a procedure for treating a valvular heart disease (VHD).

In yet another embodiment, provided herein is an assembly for preventing a heart valve leaflets prolapse, flail, or related etiology, configured to transition from an insertion configuration to a deployment configuration, the device comprising: a hollow tube defining a toroid having a plurality of apertures forming a plurality of deployment zones, the hollow tube comprising: a first end; a second end; at least one engagement mechanism coupling the first end and the second end in the deployment configuration, thereby forming a toroid defining an apical plane and a basal plane; and at least one coupling member, operable to couple an assembly-functionalizing apparatus to the toroid; and a plurality of anchors' sub-groups configured to deploy through the plurality of apertures, each anchor sub-group is being associated with, and selectably deploy through, at least one of the plurality of deployment zones.

In still yet another exemplary implementation, provided herein is an assembly for preventing prolapse, flail, billow of a heart valve leaflets, or related pathology, configured to transition from an insertion configuration to a deployment configuration, the device comprising:

a hollow tube defining a toroid having plurality of apertures forming a plurality of deployment zones, the hollow tube comprising: a first end; a second end; at least one engagement mechanism coupling the first end and the second end in the deployment configuration, thereby forming a toroid defining an apical plane and a basal plane; and a plurality of anchors' sub-groups configured to deploy through the plurality of apertures, each anchor sub-group is being associated with, and selectably deploy through, at least one of the plurality of deployment zones; a first guard member coupled to the toroid, operable to prevent a first leaflet of the heart valve leaflets from extending cephalad to a heart valve's annular plane.

In an even yet another exemplary implementation, provided herein is a kit for use in the treatment of a valvular heart disease (VHD), the kit comprising: at least one guard member for preventing prolapse, flail, or billow of at least one leaflet of a heart valve, the guard member adapted, sized and configured to operably couple to the heart valve annulus and prevent a first leaflet of the heart valve from extending cephalad to the heart valve's annular plane; packaging; and optionally instructions.

A system is provided herein for treating prolapse, flail, or billow of at least one leaflet of a native heart valve. The system may include an annuloplasty ring configured for deployment using a delivery device, the annuloplasty ring including a plurality of anchors configured to be selectively deployed to secure the annuloplasty ring to a heart valve annulus of the native heart valve, and a guard member including a plurality of struts coupled to a juncture at one end and coupled to the annuloplasty ring at another end, the guard member configured for deployment using the delivery device. The first strut and a second strut of the of plurality of struts together may extend across the annuloplasty ring to prevent prolapse, flail, or billow of the at least one leaflet of the native heart valve.

The system may further include a plurality of couplers, each coupler of the plurality of couplers coupled to a respective strut of the plurality of struts and configured to couple the respective strut to the annuloplasty ring. Each coupler of the plurality of couplers may be C-shaped. The delivery device may include a first delivery catheter configured to deliver the annuloplasty ring and a second delivery catheter configured to deliver the guard member. Each coupler of the plurality of couplers may be sized and configured to conform to a surface of the annuloplasty ring. The juncture may include a central strut. The juncture may include an intersection forming a disc shape having a central aperture. The central aperture may be configured to receive a coupling pin and the intersection may be configured to engage the guard member with the delivery device via the coupling pin. The juncture may include an intersection forming a disc shape. The intersection may be configured to be grasped by a plurality of graspers extending from the delivery device to engage the guard member with the delivery device. The guard member may be configured to transition from a restrained position corresponding to a restrained shape to an unrestrained position corresponding to an unrestrained shape. Each strut of the plurality of struts may be configured to twist about a longitudinal axis of such strut.

A method is provided herein for treating prolapse, flail, or billow of at least one leaflet of a native heart valve. The method may include causing a delivery device to deliver an annuloplasty ring at the native heart valve, the annuloplasty ring including at least one anchor, causing the annuloplasty ring to deploy the at least one anchor to secure the annuloplasty ring to a heart valve annulus of the native heart valve, causing the delivery device to deliver a guard member to the native guard valve, the guard valve including a plurality of struts coupled to a juncture at one end, and causing the delivery device to couple the guard valve to a surface of the annuloplasty ring. The first strut and the second strut of the of plurality of struts may together extend across the annuloplasty ring to prevent prolapse, flail, or billow of the at least one leaflet of the native heart valve.

The guard member may include a plurality of couplers, each coupler of the plurality of couplers coupled to a respective one the plurality of struts, and wherein causing the delivery device to couple the guard member to a surface of the annuloplasty ring comprises causing the couplers to couple to the annuloplasty ring. Each coupler of the plurality of couplers may be C-shaped. The delivery device may include a first delivery device configured to deliver the annuloplasty ring and a second delivery device configured to deliver the guard member. The juncture comprises an intersection forming a disc shape having a central aperture. The central aperture is configured to receive a coupling pin and the intersection is configured to engage the guard member with the delivery device via the coupling pin. The method may further include causing the coupling pin to disengage the intersection to release the guard member from the delivery device. The juncture comprises an intersection forming a disc shape and the intersection may be configured to be grasped by a plurality of graspers extending from the delivery device to engage the guard member with the delivery device. The method may further include causing the plurality of graspers to disengage the intersection to release the guard member from the delivery device. The method may further include causing, prior to causing the delivery device to couple the guard valve, the guard member to transition from a restrained position corresponding to a restrained shape to an unrestrained position corresponding to an unrestrained shape different from the restrained shape.

A guard member is provided herein for preventing prolapse, flail, or billow of at least one leaflet of a heart valve, and a pathology associated therewith. The guard member may be adapted, sized and configured to operably couple to the heart valve annulus and prevent a first leaflet of the heart valve from extending cephalad to the heart valve's annular plane. The heart valve annulus may further include an annuloplasty ring coupled the heart valve annulus, and wherein the guard member may be adapted sized and configured to operably couple to the annuloplasty ring. The annuloplasty ring may include a hollow tube defining a toroid having a plurality of apertures forming a plurality of deployment zones, the toroid including: a first end; a second end; at least one engagement mechanism coupling the first end and the second end in the deployment configuration; and optionally, at least one coupling member, operable to couple an assembly-functionalizing apparatus to the toroid; and a plurality of anchors' sub-groups configured to deploy through the plurality of apertures, each anchor sub-group is being associated with, and selectably deploy through, at least one of the plurality of deployment zones.

The guard member may be configured to span the annulus between two points at a predetermined position on the periphery of the annulus. The guard member may be a tri-rod defining three ends, each end configured to engage the annulus at a predetermined position on the periphery of the annulus, operable to prevent the heart valve leaflets prolapse, flail or other related etiology. The guard member may include a coil configured to engage the annulus forming a layer. The coil forms a cone having a caudal apex. An extent to which the apex extends caudal may be adjustable. The guard member may further include another guard member, coupled to the annulus, operable to prevent a second leaflet of the heart valve leaflets from extending cephalad to a heart valve's annular plane. The guard member may further include another guard member, coupled to the annulus, operable to prevent a second leaflet of the heart valve leaflets from extending cephalad to a heart valve's annular plane. At least one of: the guard member, and the other guard member, each may be covered with a sleeve configured to at least one of: prevent the formation of a thrombus on the at least one of: the guard member, and the other guard member; and promote ingrowth of tissue onto the first end of the guard member, and/or the first end of the second guard member.

At least one of: the guard member, the other guard member, and the annuloplasty ring, each may be covered with a sleeve configured to at least one of: prevent the formation of a thrombus on the at least one of: the guard member, the other guard member, and the annuloplasty ring; and promote ingrowth of tissue onto the first end of the guard member, and/or the first end of the second guard member, and/or the annuloplasty ring. The sleeve may include a biocompatible fabric impregnated with an agent configured to prevent formation of thrombocytes. The related pathology may be at least one of: an endocarditis, a ruptured chordac, a myxomatously degenerated valve, a blunt chest trauma, and a congenital defect. The heart valve may be: a mitral valve, a tricuspid valve or both the mitral valve and the tricuspid valve.

Use of a first guard member is provided herein. The first guard member may be configured to transition from an insertion configuration to a deployment configuration, the first guard member adapted sized and configured to be coupled to a heart valve annulus, operable to prevent a first leaflet of the heart valve leaflets from extending cephalad to the heart valve's annular plane, in a procedure for treating a valvular heart disease (VHD). The valvular heart disease is at least one of Mitral valve prolapse (MVP), Mitral Valve flail, floppy mitral valve syndrome, systolic click-murmur syndrome, billowing mitral leaflets, tricuspid valve prolapse, Ehlers-Danlos syndrome, Marfan's syndrome (MFS), Ebstein's anomaly, septum secundum (ASD), ruptured or overextended chordae and any other disease causing any leaflet to extend cephalad to the heart valve's annular plane. The heart valve annulus further including an annuloplasty ring coupled the heart valve annulus, and wherein the first guard member is adapted, sized, and configured to operably couple to the annuloplasty ring.

The annuloplasty ring may include a hollow tube defining a toroid having a plurality of apertures forming a plurality of deployment zones. The toroid may include a first end; a second end; at least one engagement mechanism coupling the first end and the second end in the deployment configuration; and optionally, at least one coupling member, operable to couple an assembly-functionalizing apparatus to the toroid; and a plurality of anchors' sub-groups configured to deploy through the plurality of apertures, each anchor sub-group is being associated with, and selectably deploy through, at least one of the plurality of deployment zones. The first guard member defines a first end configured to engage the annulus in its deployed configuration.

The first guard member defines a first end configure to engage the toroid in its deployed configuration. The first guard member may be configured to span the annulus between two points at a predetermined position on the periphery of the annulus. The first guard member may be configured to span the annuloplasty ring between two points at a predetermined position on the periphery of the annuloplasty ring. The first guard member may be a tri-rod defining three ends, each end configured to engage the annulus at a predetermined position on the periphery of the annulus, operable to prevent the heart valve leaflets prolapse, flail or related etiology.

The first guard member may be a tri-rod defining three ends, each end configured to engage the annuloplasty ring at a predetermined position on the periphery of the annuloplasty ring, operable to prevent the heart valve leaflets prolapse, flail or related etiology. The first guard member may include a planar coil configured to engage the annulus forming a layer. The coil may form a cone having a caudal apex. The first guard member may include a planar coil configured to engage the annuloplasty ring forming a layer. The coil may forms a cone having an adjustable caudal apex. A second guard member, coupled to the annulus, may be operable to prevent a second leaflet of the heart valve leaflets from extending cephalad to the heart valve's annular plane. A second guard member may be coupled to the annuloplasty ring and may be operable to prevent a second leaflet of the heart valve leaflets from extending cephalad to the heart valve's annular plane. At least one of: the first guard member, and the second guard member, may each be covered with a sleeve configured to at least one of: prevent the formation of a thrombus on at least one of: the guard member, and the other guard member; and promote ingrowth of tissue onto the first end of the guard member, and/or the other guard member. At least one of: the first guard member, and the second guard member, may each be covered with a sleeve configured to at least one of: prevent the formation of a thrombus on at least one of: the guard member, and the other guard member; and promote ingrowth of tissue onto the first end of the guard member, and/or the other guard member. The sleeve may include a biocompatible fabric impregnated with an agent configured to prevent formation of thrombocytes. The related pathology may be at least one of: an endocarditis, a ruptured chorda, a myxomatously degenerated valve, a blunt chest trauma, and a congenital defect. The heart valve may be at least one of: a mitral valve, a tricuspid valve or both the mitral valve and the tricuspid valve.

An assembly is provided herein for preventing a heart valve leaflets prolapse, flail, or related etiology, configured to transition from an insertion configuration to a deployment configuration. The assembly device may include a hollow tube defining a toroid having a plurality of apertures forming a plurality of deployment zones, the hollow tube including: a first end; a second end; at least one engagement mechanism coupling the first end and the second end in the deployment configuration, thereby forming a toroid defining an apical plane and a basal plane; and at least one coupling member, operable to couple an assembly-functionalizing apparatus to the toroid; and a plurality of anchors' sub-groups configured to deploy through the plurality of apertures, each anchor sub-group is being associated with, and selectably deploy through, at least one of the plurality of deployment zones.

An assembly is provided herein for preventing prolapse, flail, billow of a heart valve leaflets, or related pathology. The assembly may be configured to transition from an insertion configuration to a deployment configuration. The assembly device may include a hollow tube defining a toroid having plurality of apertures forming a plurality of deployment zones, the hollow tube including: a first end; a second end; at least one engagement mechanism coupling the first end and the second end in the deployment configuration, thereby forming a toroid defining an apical plane and a basal plane; and a plurality of anchors' sub-groups configured to deploy through the plurality of apertures, each anchor sub-group is being associated with, and selectably deploy through, at least one of the plurality of deployment zones; a first guard member coupled to the toroid, operable to prevent a first leaflet of the heart valve leaflets from extending cephalad to a heart valve's annular plane. The guard member and the annuloplasty ring may be operable to be delivered using a single delivery catheter. Alternatively, the guard member may be operable to be delivered using a first delivery catheter, separate from a second catheter used to deliver the annuloplasty ring.

A kit is provided herein for use in the treatment of a valvular heart disease (VHD). The kit may include at least one guard member for preventing prolapse, flail, or billow of at least one leaflet of a heart valve, the guard member adapted, sized and configured to operably couple to the heart valve annulus and prevent a first leaflet of the heart valve from extending cephalad to the heart valve's annular plane; packaging; and optionally instructions. The kit may further include at least one annuloplasty ring. The at least one annuloplasty ring may include a hollow tube defining a toroid having a plurality of apertures forming a plurality of deployment zones, the toroid including: a first end; a second end; at least one engagement mechanism coupling the first end and the second end in the deployment configuration; and optionally, at least one coupling member, operable to couple an assembly-functionalizing apparatus to the toroid; and a plurality of anchors' sub-groups configured to deploy through the plurality of apertures, each anchor sub-group is being associated with, and selectably deploy through, at least one of the plurality of deployment zones. The guard member may define a first end configure to engage the annulus in its deployed configuration. The guard member may defines a first end configure to engage the annuloplasty ring in its deployed configuration. The first guard may be configured to span the annulus between two points at a predetermined position on the periphery of the annulus.

The first guard may be configured to span the annuloplasty ring between two points at a predetermined position on the periphery of the annuloplasty ring. The guard member may be a tri-rod defining three ends, each end configured to engage the annulus at a predetermined position on the periphery of the annulus, operable to prevent the heart valve leaflets prolapse, flail or related etiology. The guard member may be a tri-rod defining three ends, each end configured to engage the annuloplasty ring at a predetermined position on the periphery of the annuloplasty ring, operable to prevent the heart valve leaflets prolapse, flail or related etiology. The guard member may include a planar coil configured to engage the annulus forming a layer. The coil may form a cone having an adjustable caudal apex. The guard member may include a planar coil configured to engage the annuloplasty ring forming a layer. The coil may form a cone having an adjustable caudal apex. The kit may include a plurality of guard members of the same configuration having a different size. The kit may include a plurality of guard members having different configuration, each configuration having the same or different size.

The kit may include a plurality of guard members, each guard member having the same configuration with a different size; and a plurality of annuloplasty rings having the same configuration with a different size. The kit may include a plurality of guard members, each guard member having different configuration, with each configuration having the same, or different size; and a plurality of annuloplasty rings having the same configuration with a different size. The kit may include a delivery catheter. The kit may include a single delivery catheter configured to transition the annuloplasty ring and the guard member from an insertion configuration to a deployed configuration. The kit may include a first delivery catheter configured to transition the annuloplasty ring from an insertion configuration to a deployed configuration; and a second delivery catheter configured to transition the guard member from an insertion configuration to a deployed configuration.

### BRIEF DESCRIPTION OF THE DRAWINGS

The method implementable using the alignment and engagement systems disclosed herein will become apparent from the following detailed description when read in conjunction with the figures, which are exemplary, not limiting, and in which:

FIG. 1A, illustrates top plan view of a first exemplary implementation of the assembly for preventing a prolapse, flail, or billow of heart valve leaflet(s), with FIG. 1B illustrating a front perspective view thereof;

FIG. 2A, illustrates top plan view of a second exemplary implementation of the assembly for preventing a prolapse, flail, or billow of heart valve leaflet(s), with FIG. 2B illustrating a perspective view thereof;

FIG. 3A, illustrates top plan view of a third exemplary implementation of the assembly for preventing a prolapse, flail, or billow of heart valve leaflet(s), with FIG. 3B illustrating a perspective view thereof;

FIG. 4A, illustrates top plan view of a fourth exemplary implementation of the assembly for preventing a prolapse, flail, or billow of heart valve leaflet(s), with FIG. 4B illustrating a perspective view thereof;

FIG. 5, illustrate another exemplary implementation of the guard member component of the assembly, configured for insertion in a delivery catheter, shown in its expanded deployed configuration;

FIG. 6A is a schematic illustration of an exemplary implementation of the delivery catheter used to couple a guard member onto the annuloplasty platform ring, with FIG. 6B being an enlargement of circled area A in FIG. 6A, further illustrated in FIG. 6C, while FIG. 6D illustrates the distal end of the delivery catheter following deployment and coupling; and

FIG. 7A, is a schematic illustration of another exemplary implementation of the distal end of the delivery catheter illustrated in FIG. 6A, used to couple another exemplary implementation of the guard member onto the annuloplasty platform ring, with FIG. 7B being an enlargement of circled area B in FIG. 7A, further illustrated in FIG. 7C, while FIG. 7D illustrates the distal end of the delivery catheter following deployment and coupling.

While the disclosure of the assemblies for preventing a heart valve leaflets prolapse, flail, and/or billow and their use, is amenable to various modifications and alternative forms, specifics thereof have been shown by way of example in the drawings and will be further described in detail hereinbelow. It should be understood, however, that the intention is not to limit the disclosure to the particular exemplary implementations described. On the contrary, the intention is to cover all modifications, equivalents, and alternatives.

### DETAILED DESCRIPTION

Provided herein are exemplary implementations of guard member for preventing prolapse, flail, or billow of at least one leaflet of a heart valve and a pathology associated therewith, the guard member adapted, sized and configured to operably couple to the heart valve annulus and prevent a first leaflet of the heart valve from extending cephalad to the heart valve's annular plane.

Moreover, provided herein are guard member adapted, sized and configured to operably couple to the heart valve annulus and prevent a first leaflet of the heart valve from extending cephalad to the heart valve's annular plane, use of these guards and kits containing one or more types of the guards.

### Definitions

The term “coupled”, including its various forms such as “operably coupling”, “coupling” or “couplable”, refers to and comprises any direct or indirect, structural coupling, connection or attachment, or adaptation or capability for such a direct or indirect structural or operational coupling, connection or attachment, including integrally formed components and components which are coupled via or through another component or by the forming process. Indirect coupling may involve coupling through an intermediary member or adhesive, or abutting and otherwise resting against, whether frictionally or by separate means without any physical connection.

In addition, for the purposes of the present disclosure, directional or positional terms such as “top”, “bottom”, “upper,” “lower,” “side,” “front,” “frontal,” “forward,” “rear,” “rearward,” “back,” “trailing,” “above,” “below,” “left,” “right,” “radial,” “vertical,” “upward,” “downward,” “outer,” “inner,” “exterior,” “interior,” “intermediate,”, “apical”, “basal”, etc., are merely used for convenience in describing the various exemplary implementations of the present disclosure.

Likewise, the term “engage” and various forms thereof, when used with reference to an engaging element, for example in the engagement of the first guard member 110 and its at least two interposers (e.g., 1101, 1102), each of the interposers configured to engage toroid 100 at a predetermined position on the periphery of toroid 100, refers in an exemplary implementation to the application of any forces that tend to hold guard member 110 and the pair (or more) of interposers together against inadvertent or undesired separating forces (e.g., such as may be introduced during alignment/engagement and manipulation of the annuloplasty ring platform or its' toroidal portion). It is to be understood, however, that engagement does not in all cases require an interlocking connection that is maintained against every conceivable type or magnitude of separating force. Further, the term “engaging element” refers in another exemplary implementation to one or a plurality of coupled components, at least one of which is configured for releasably engaging another element. Thus, this term encompasses both single part engaging elements and multi-part-assemblies, for example coupling assembly 303 as a whole.

The terms “first,” “second,” and the like, herein do not denote any order, quantity, or importance, but rather are used to denote one element from another. The terms “a”, “an” and “the” herein do not denote a limitation of quantity, and are to be construed to cover both the singular and the plural, unless otherwise indicated herein or clearly contradicted by context. The suffix “(s)” as used herein is intended to include both the singular and the plural of the term that it modifies, thereby including one or more of that term (e.g., lance-member(s) 2014j includes one or more lance member).

Reference throughout the specification to “one exemplary implementation”, “another exemplary implementation”, “an exemplary implementation”, and so forth, means that a particular element (e.g., step, feature, structure, and/or characteristic) described in connection with the exemplary implementation is included in at least one exemplary implementation described herein, and may or may not be present in other exemplary implementations. In addition, it is to be understood that the described elements may be combined in any suitable manner in the various exemplary implementations.

In the context of the disclosure, the term “operable” means the system and/or the device, or a certain element or step is fully functional, sized, adapted and calibrated, comprises elements for, and meets applicable operability requirements to perform a recited function when activated, coupled, implemented, actuated, effected, or realized. In relation to systems, the term “operable” means the system is fully functional and calibrated, having the necessary elements, as well as the mechanisms for, and meets applicable operability requirements to perform a recited function when executed by a user.

In the context of the disclosure, the term “saddle-shaped” is used herein to mean an annuloplasty ring generally made of two arcuate members for example, two toroidal portions with each toroidal portion having an apex and two ends connecting the toroidal portions. The apex of the toroidal portion in one member can be in the same or opposite direction of the other member. The formed ring can be generally D-shaped.

In the context of the disclosure, “Mitral/Tricuspid valve prolapse” refers to the displacement of any leaflet tissue cephalad (towards the head) past the heart-valve's annular plane. In certain cases, the valve leaflets either detach from the chordae tendinae, the structure that tethers them to the ventricular wall so that they are positioned to coapt or close against the other valve leaflet during systole, or the chordae tendinae elongates to the point of being ineffective. In this case, the leaflet “flails” (if the whole leaflet) or billows (if portion thereof only) instead of coapting or sealing against the neighboring leaflet allowing blood to surge into the wrong heart chamber.

In the context of the disclosure, the term “cephalad” refers to the area extending between the annular plane of the relevant heart valve and the head of the subject, where conversely, the term “caudal” refers to the area extending between the annular plane of the relevant heart valve and the tail (or base of the spine) of the subject.

In the context of the disclosure, the term “strained” as used to describe the strained guard member means that the guard member is maintained under an extrinsic tensile strain. An “extrinsic strain” as used herein refers to a tensile strain that is applied to the guard member by the deployment module of the delivery catheter (or an external force), rather than a tensile strain developed within the strained guard member. In other words, in the unstrained position, the guard member, formed of a resilient material (e.g., nitinol, stainless steel and the like) curls and/or twists, and the insertion into the manipulation module causes the tensile stress to form the strain imposed on the guard member.

A more complete understanding of the guard member adapted, sized and configured to operably couple to the heart valve annulus and prevent a first leaflet of the heart valve from extending cephalad to the heart valve's annular plane whether or not coupled to an annuloplasty ring assembly having an adapter for, or an integrated structurally with guard member, can be obtained by reference to the accompanying drawings. These figures (also referred to herein as “FIG.”) are merely schematic representations based on convenience and the case of demonstrating the present disclosure, and are, therefore, not intended to indicate relative size, scale and dimensions of the devices or components thereof, and/or to define or limit the scope of the exemplary implementations. Although specific terms are used in the following description for the sake of clarity, these terms are intended to refer only to the particular structure of the exemplary implementations selected for illustration in the drawings, and are not intended to define or limit the scope of the disclosure. In the drawings and the following description below, it is to be understood that like numeric designations refer to components of like function.

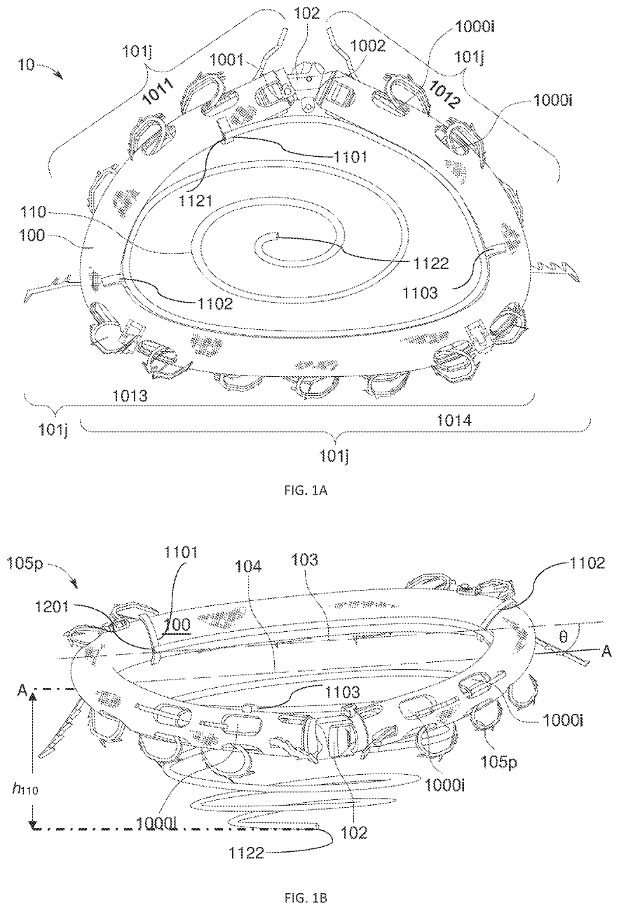

Turning now to FIGS. 1A-4B, illustrating exemplary implementations of the prolapse/flail/billow of leaflet(s) cephalad to the heart valve's annular plane. As illustrated, provided is guard member 110 for preventing prolapse, flail, or billow of at least one leaflet of a heart valve, and a pathology associated therewith, guard member 110 adapted, sized and configured to operably couple to the heart valve annulus and prevent a first leaflet of the heart valve from extending cephalad to the heart valve's annular plane. In certain exemplary implementations, and as further illustrated in FIGS. 1A, 1B, the heart valve annulus (not shown) can further comprise an annuloplasty ring 100 coupled the heart valve annulus, and wherein the guard member 110 is adapted sized and configured to operably couple to the annuloplasty ring.

Turning now to FIG. 1A, illustrating top view of assembly 10. Assembly 10 can include annuloplasty ring 100 that defines the shape of ring assembly 10. Annuloplasty ring 100 can include one or more apertures 1000i about its circumference. One or more anchors 105p can be positioned and configured to extend through apertures 1000i. Ring assembly (or platform) 10 can also include number of zones 101j with corresponding anchors 105p configured to selectably extend radially (as well as in ventral and dorsal direction, see e.g. ang θ, FIG. 1B). As illustrated, e.g., in FIGS. 1A, 2A, 3A, and 4A there are, in certain exemplary implementations, four deployment zones 101j, two anterior zones 101j, 1011, 1012, and two posterior zones 101j, 1013, 1014, which overlap.

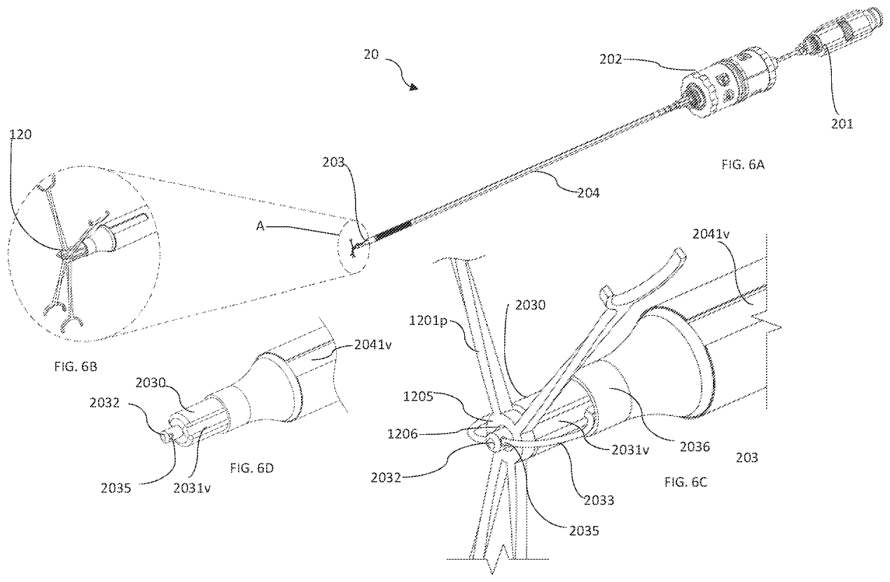

Also illustrated is engagement mechanism 102. Engagement mechanism 102 can be configured to lock second end 1002 of ring assembly (or platform) 10 to first end 1001 of ring assembly 10. Engagement mechanism 102 can also be designed and configured to be removeably attached to delivery catheter (not shown) for assembly 10. For example, in certain implementations, engagement mechanism 102 can include pivot pin (not shown) that is configured to removeably attach ring assembly (or platform) 10 to delivery device as well as provide for rotation of ring assembly (or platform) 10 when expelled from delivery device (not shown, for exemplary implementation of delivery device, or catheter, see e.g., FIG. 6A). For example, pivot pin can be configured to provide for 90 degrees of rotation of ring assembly (or platform) 10. In other implementations, pivot pin can be configured to provide for additional ranges of rotation such as 75-105 degrees of rotation, 60-120 degrees of rotation, and other similar ranges of rotation.

Accordingly and in an exemplary implementation, the annuloplasty ring 100 comprises: hollow tube 103 (not shown, under mesh illustrated in FIGS. 1A-4B) defining a toroid having plurality of apertures 105p forming plurality of deployment zones, toroid 103 comprising: first end 1001; second end 1002; at least one engagement mechanism 102 coupling first end 1001 and second end 1002 in deployment configuration; and optionally, at least one coupling member 1101, operable to couple assembly-functionalizing apparatus 110 to toroid; and plurality of anchors' sub-groups 101j configured to deploy through plurality of apertures 105p, each anchor sub-group 101j is being associated with, and selectably deploy through, at least one of plurality of deployment zones.

In an exemplary implementation, and as illustrated in FIGS. 1A-4B, guard member 110 is configured to span the annulus between two or more points at a predetermined position on the periphery of the annulus. Those points can be associated with natural fiducials in the heart valve, such as the mid-line of a leaflet, a coaptation seam, and the like. For example, guard member 110 is a tri-rod defining three ends, each end configured to engage the annulus (either directly, or operably coupled to annuloplasty ring 100) at a predetermined position on the periphery of the annulus, guard member 110 being operable (in other words, positioned properly and in a therapeutically effective manner), to prevent the heart valve leaflet(s) or cusp(s) prolapse, flail, or other related etiology.

In an exemplary implementation of the guard member for preventing prolapse, flail, or billow of at least one leaflet of a heart valve, and a pathology associated therewith, the “related pathology” can be at least one of: an endocarditis (a symptom of prolonged prolapse, flail, billow or their combination), a ruptured chordac, a myxomatously degenerated valve, a blunt chest trauma, a congenital defect, or any permutation of a combination of the foregoing pathologies. For example, blunt chest trauma can lead to torn chordate, which in turn can lead to prolapse, flail or billowing of tricuspid valve cusp, expressed as sometimes-severe tricuspid regurgitation. Likewise primary mitral leaflet billow, with or without prolapse, is typically associated with myxomatous degeneration of the mitral valve apparatus, mainly the posterior leaflet, exhibiting clinically as an isolated nonejection systolic click (billow), a murmur of mitral regurgitation that is usually late systolic (prolapse), or a combination of murmur and click. In certain exemplary implementations, guard members 110 can be used to arrest the billowing, flail or prolapse described. Accordingly, guard member can be used wherein the heart valve is: a mitral valve, a tricuspid valve or both the mitral valve and the tricuspid valve.

As illustrated in FIG. 1A, and in an exemplary implementation, guard member 110 is a coil having a first end 1201, coupled to the toroid via first coupler 1101. Additional couplers 1100n (1102, 1103) can be used to couple guard member 110 to annuloplasty ring 100. External portion of the coil can be configured to abut the internal surface of annuloplasty ring 100, while the second end 1122 of the coil forming guard member 110 can extend caudally and be adjustable relative to the annular plane of the valve, as well be further adjusted relative to the flailing/billowing leaflet, and the annular valve plane. As further illustrated in FIG. 1B, guard member 110 coil forms a cone having a caudal (extending downward) apex, defining height h₁₁₀, between second end 1122 of the coil (110—guard member), and plane A-A (see e.g., FIG. 1B) formed by the dorsal portion of annuloplasty ring 100. That height h₁₁₀, can be adjusted and its angle changed, depending on the orientation of annuloplasty ring 100 relative to the plane of the heart valve annulus, and the type and location of the prolapsing, flailing or billowing leaflet or cusp.

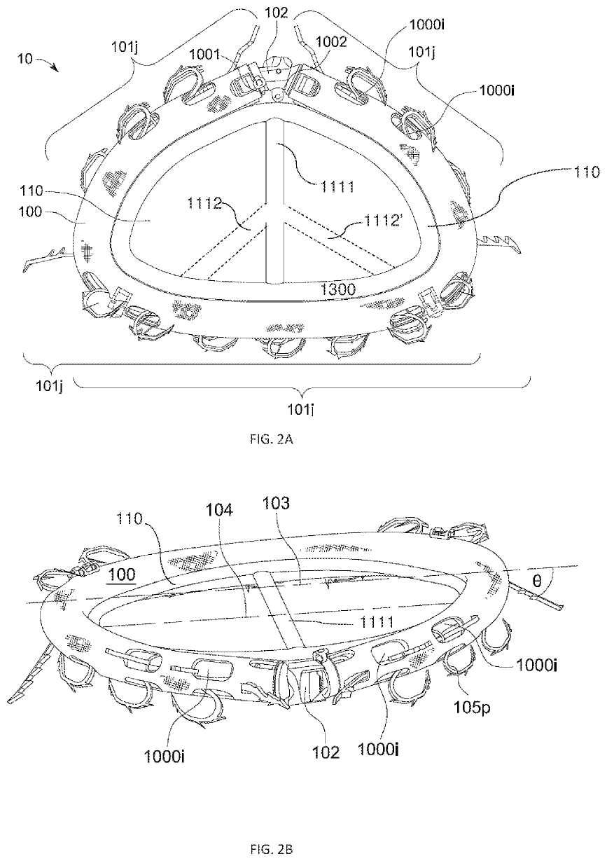

Turning now to FIGS. 2A, 2B, illustrating another exemplary implementation of the guard member operable for preventing prolapse, flail, or billow of at least one leaflet, or cusp of a heart valve, and a pathology associated therewith, where guard member 110 comprises a peripheral section 1300, configured to be complimentary to the internal surface of annuloplasty ring 100 (or the heart-valve annulus), and define a plane that is parallel or matches that of the corresponding heart valve annular plane. In other words, the plane defined can be saddle shape, concave, convex, planar, D-shaped, or a combination thereof. In certain exemplary implementations, the first guard 110 having at least one spanner 1111 is configured to span the toroid between two (or more) points (in other words, forming a chord) at a predetermined position on the periphery of the toroid. Spanner 1111 can also comprise additional extensions 1112, 1113.

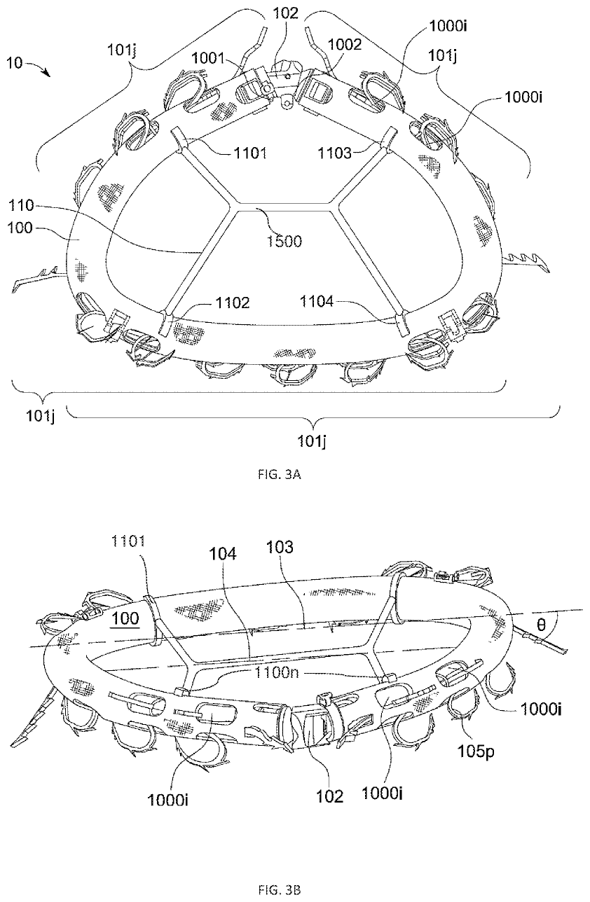

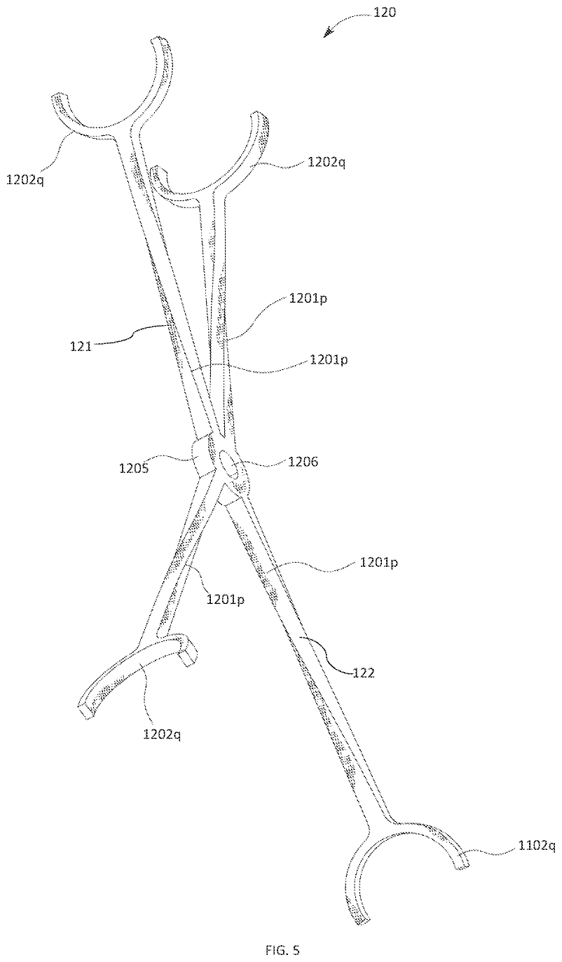

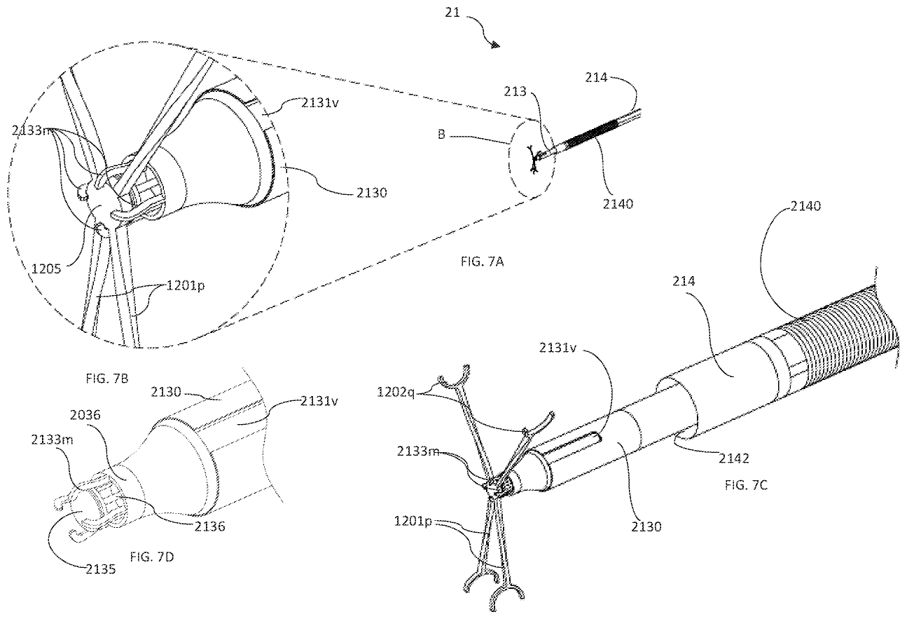

Turning now to FIGS. 3A, 3B, illustrating another exemplary implementation of guard member 110, having plurality (e.g., four) points of contact with toroid tube 110, extending at the ends of extension from central span 1500. Central span 1500 may be a mid-section connecting plurality of struts (e.g., four) to one another. For example, central span 1500 may be a juncture such as a central strut connecting each strut to each other strut. Central span may be a strut (e.g., member, bar, etc.), a circular structure (e.g., the circular structure illustrated in FIG. 5 or disc illustrated in FIGS. 7A-7D), and/or may form any suitable shape. While four struts are illustrated in FIGS. 3A-3B, it is understood that greater than four (e.g., 2 or 3) or less than four (e.g., 5, 6, 7, 8, etc.) may be used. While each strut is shown having a generally linear shape, it is understood that one or more strut may have a curved or other shape.

Each strut may connect to a respective coupler (e.g., couplers 1100n). Each coupler may have a C-shape and/or may conform to the surface of annuloplasty ring 100 and/or toroid tube 110. In one example, annuloplasty ring 100 may have a circular cross-sectional profile and the coupler may be C-shaped and/or may be designed to snap-fit together with the annuloplasty ring. Alternatively, each coupler may have a different shape designed to connect with and secure guard member 110 to annuloplasty ring 100.

Guard member 110 may be sized such that, when guard member 110 is in the unrestrained position and engaged with annuloplasty ring 100, guard member 100 has a length that is the same as the distance spanning a void defined by the toroidal shaped annuloplasty ring. In another example, guard member 101 may be sized such that, when guard member 110 is in the unrestrained position, guard member 100 has a length greater than a distance spanning across the void defined by the toroidal shaped annuloplasty ring. When the annuloplasty ring is engaged with the guard member, a load may be applied by the guard member to the annuloplasty ring due to the size of the guard member. In this example, the guard member may be secured to the ring by the couplers as well as the geometry of the guard member and/or the force applied by the guard member to the annuloplasty ring.

While guard member 100 is secured to annuloplasty ring, struts may together extend across the annuloplasty ring to interface with nature leaflets to prevent prolapse, flail, or billow of at least one leaflet of the heart valve. For example, two struts joined at the juncture (e.g., central strut) may interface with the leaflet to prevent billowing of the leaflet.

As illustrated, couplers 1100n (1101, 1102, 1103, 1104) may not be positioned equidistant from central span 1100 and are configured to prevent prolapse, billow or flail of a heart-valve leaflet(s) or cusp(s) that may not be the same size. For example, guard member 110 may include struts having different lengths and/or sizes. Additionally, or alternatively, struts of guard member 110 may have a uniform cross-section and/or may have a cross-section that varies in shape and/or size.

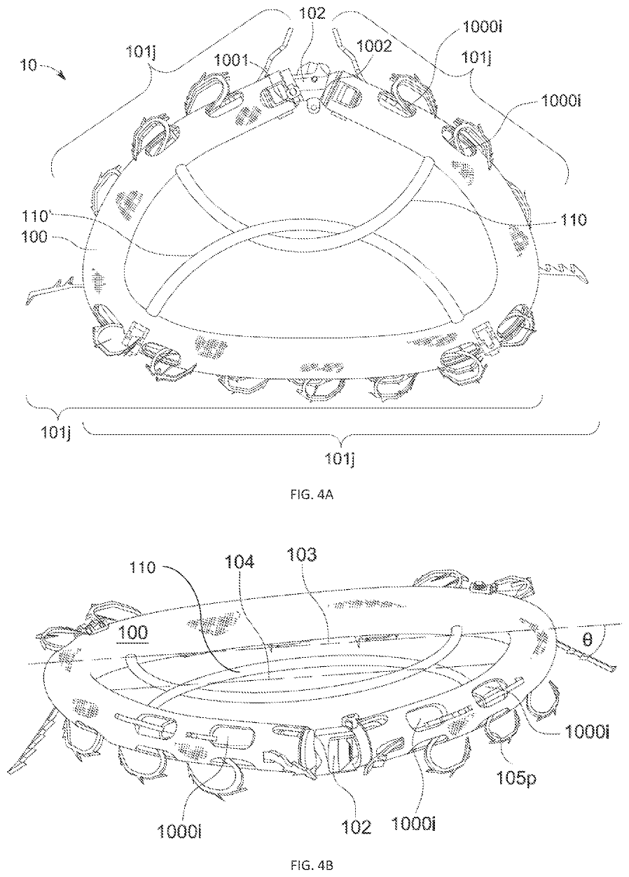

Similarly, FIGS. 4A, 4B, illustrate arcuate first guard member 110, integrated with annuloplasty ring 100, with interlaced second guard member 110′ also integrated to annuloplasty ring 100, whereby first guard member 110 and second guard member 110′ can have the same or different segment size enclosed by the respective guard member 110, 110′.

Accordingly and in certain exemplary implementations, annuloplasty ring 100, or, if coupled directly, the heart-valve annulus can further comprise another (two or more) guard member 110′, coupled to the annulus, operable to prevent a second (or third) leaflet (or cusp) of the heart valve from extending cephalad to the heart valve's annular plane. It is further noted, that in the context of the disclosure, the term “heart valve's annular plane” does NOT necessarily mean a flat plane, but as indicate herein, the heart valve's annular plane can be can be saddle shape, concave, convex, planar, D-shaped, or a combination thereof.

Further, in certain exemplary implementation, at least one of: the first guard member 110, and the second guard member 110′, is covered with a sleeve configured to at least one of: prevent the formation of a thrombus (e.g., due to pulsatile and turbulent blood flow across the guard member(s)) on at least one of the first guard member, and the toroid; and promote ingrowth of tissue onto the first end or into any couplers 1100n of any guard member integrated with, or coupled to the annuloplasty ring 100. Accordingly, the sleeve comprises a biocompatible fabric impregnated with an agent configured to prevent formation of thrombocytes. For example, the polymeric material can be, for example: polyethylene terephthalate (PET), naphthalene dicarboxylate derivative polymer, configured to have mechanical and chemical properties including anti-thrombocyte forming, a radiation resistant and hydrolytically stable biocompatible fabric having a textile construction of a plurality of polymeric filaments. These filaments can be bundled into yarns, which can then be woven, braided, knitted, or otherwise combined into a textile fabric. Furthermore, in certain exemplary implementations and as illustrated in FIGS. 1A-4B the fabric sleeve is used to cover annuloplasty ring 100 as well.

Turning now to FIGS. 5-6B, illustrating an exemplary implementation of guard member 120, configured for insertion in a dedicated delivery catheter 200 (see e.g., FIG. 6A), shown in its expanded deployed configuration. As illustrated, guard member 120, is configured to transition from insertion configuration to deployed configuration when expelled beyond a certain distance distally from collar 2036 by pushing of shaft 2030.

As illustrated, guard member 120 may be the same as or similar to guard member 110 in FIGS. 3A-3B. For example, guard member 120 has a general X-shape with members 1201p, which each may be struts, extending from central intersection 1205, defining aperture 1206 each member 1201p terminating in coupler 1200n (1201, 1202, 1203, 1204). For example, central intersection 1205 may have a circular or disc shape with aperture 1206 positioned in the middle. As indicated, in this exemplary implementation, guard member 120 is configured to transition between a strained position inside delivery catheter 200 lead tube 204, when delivered to annuloplasty ring 100 (already coupled to the heart valve annulus), and an unstrained position as illustrated in FIG. 6C.

As illustrated, each member 1201p may be formed from a shape memory material that may be designed to transition from a restrained position having a restrained shape to an unrestrained position having an unrestrained shape that is different from the restrained shape. For example, each member may have an unrestrained position having a twist along a longitudinal axis of member 1201p. Member 1201p may further have a restrained position without such twist or may be twisted in the opposite direction.

The torsional orientation of member 1201p in the unrestrained position may cause member 1201p to extend outwardly away from a neighboring member. Prior to deployment, delivery catheter 200 may maintain member 1201p in the restrained position within delivery catheter 200. In the restrained position, member 1201p may be closer to a neighboring member than in the restrained position and/or may be restrained such that guard member 110 is positioned within delivery catheter 200.

As further illustrated in FIG. 6B, guard member 110, once in the unstrained position, is configured to form a substantially X shaped member with member extensions 1201p not necessarily being equidistant from intersection 1205. Guard member 110, having members 1201p extending from intersection 1205 also transition from strained position (e.g., restrained position) where couplers 1200n are configured from a first position before deployment and rotate axially post deployment at predetermined position 121, 122 along each extension member 1201p. For example, as guard member 120 is deployed by delivery catheter 200, guard member may transition from the restrained position to the unrestrained position and may rotate axially from a delivery orientation to a deployment orientation.

As illustrated in FIG. 6A, delivery catheter 200 is comprised of a manipulation module 201, drivetrain 202, guide tube 204 and deployment end module 203 (see e.g., FIGS. 6C, 6D, 7C, 7D). As illustrated in FIG. 6C, deployment module 203 can be comprised of shaft 2030, defining therein channels 2031v configured to accommodate extensions 1201p while in the insertion configuration. Also illustrated is coupling pin 2032 defining bore 2035 configured to allow passage of release wire 2033, extending all the way back to the proximal end in manipulation module 201. Coupling pin may extend through intersection 1205 of guard member 120, which may form a juncture for each member. Intersection 1205 may be a juncture connecting each strut to each other strut. Release wire 2033 is maintained such it limits rotation of guard member 120 about coupling pin 2032, whereby upon engaging of annuloplasty ring 100 as desired using couplers 1200n, release wire is pulled, releasing guard member 120 from delivery catheter 200.

Yet another exemplary implementation is illustrated in FIGS. 7A-7D. In this example, guard member 120 intersection 1205 does not define aperture 1206, but rather forms a disc that is held in place by rod 2136 having distal flange 2135 which holds intersection 1205 disc against resilient graspers 2133m, configured as illustrated in FIG. 7B, to prevent the rotation of guard member 120 during the coupling of guard member 120 to annuloplasty ring 100. Intersection 1205 forming a disc may be a juncture connecting each strut to each other strut While four graspers 2133m are illustrated in FIG. 7B, it is understood that fewer than four (e.g., 2 or 3) or greater than four (e.g., five, six, etc.) graspers may be used. Following coupling and engagement of guard member and annuloplasty ring 100, rod 2136 is pushed forward (distally), pushing intersection 1205 disc beyond graspers 2133m and releasing guard member 120 from delivery catheter 200.

In another example, the guard member illustrated in FIGS. 3A-3B may be used and one or more suitable graspers may be used to secure the central span (e.g., central span 1500 of FIGS. 3A-3B) to the deployment catheter. The guard member illustrated in FIGS. 3A-3B may similarly transition from a restrained position to an unrestrained position. For example, the guard member may be introduced to a deployment site in a restrained position connected to the delivery catheter and may be permitted to transition to the unrestrained position when released by the delivery catheter.

Guard member as illustrated, may be coated with fabric as disclosed herein and be configured for direct coupling to the heart valve annulus rather than to annuloplasty ring 100. Under these circumstances, couplers 1202q (see e.g., FIG. 7C) can be equipped with proper anchoring hooks, harpoons and the like to ensure secure coupling to the heart valve annulus.

In an exemplary implementation, the methods and uses disclosed herein, are implemented using the devices and systems disclosed herein. Accordingly, provided herein is use of the assemblies and guard members disclosed herein and their equivalents and variations in a procedure for treating a valvular heart disease. For example, the valvular heart disease is at least one of Mitral valve prolapse (MVP), floppy mitral valve syndrome, systolic click-murmur syndrome, billowing mitral leaflets, tricuspid valve prolapse, Ehlers-Danlos syndrome, Marfan's syndrome (MFS), Ebstein's anomaly, septum secundum (ASD), and ruptured or overextended chordac.

Furthermore, in an exemplary implementation, provided herein is an assembly for preventing a heart valve leaflet(s) prolapse, flail, billow or related pathology, configured to transition from an insertion configuration to a deployment configuration, the device comprising: a hollow tube defining a plurality of apertures forming a plurality of deployment zones, the hollow tube comprising: a first end; a second end; at least one engagement mechanism coupling the first end and the second end in the deployment configuration; and at least one coupling member, operable to couple an assembly-functionalizing apparatus to the toroid; and a plurality of anchors' sub-groups configured to deploy through the plurality of apertures, each anchor sub-group is being associated with, and selectably deploy through, at least one of the plurality of deployment zones, while in another exemplary implementation, provided herein is an assembly for preventing prolapse, flail, billow of a heart valve leaflet(s) or related pathology, configured to transition from an insertion configuration to a deployment configuration, the device comprising: a hollow tube defining a plurality of apertures forming a plurality of deployment zones, the hollow tube comprising: a first end; a second end; at least one engagement mechanism coupling the first end and the second end in the deployment configuration, thereby forming a toroid defining an apical plane and a basal plane; and a plurality of anchors' sub-groups configured to deploy through the plurality of apertures, each anchor sub-group is being associated with, and selectably deploy through, at least one of the plurality of deployment zones; a first guard member coupled to the toroid, operable to prevent a first leaflet of the heart valve leaflets from extending cephalad to a heart valve's annular plane.

It is contemplated that a physician may have different guard members as illustrated in FIGS. 1A-5, each which can be used depending on the pathology sought to be addressed. Accordingly and in yet another exemplary implementations, the guard members 110 disclosed herein are used as part of a kit, and thus provided herein is a kit for use in the treatment of a valvular heart disease (VHD), the kit comprising: at least one guard member for preventing prolapse, flail, or billow of at least one leaflet of a heart valve, the guard member adapted, sized and configured to operably couple to the heart valve annulus and prevent a first leaflet of the heart valve from extending cephalad to the heart valve's annular plane; packaging; and optionally instructions. Furthermore, the kit can further comprise one or more annuloplasty rings, each of different size, or sized and configured for a different heart valve. It is further contemplated, that the kit may comprise different types of guard members, identical to, or similar to those guard members illustrated in FIGS. 1A-7D. Likewise, the kit may further comprise at least one delivery catheter, identical to or operationally equivalent to the delivery catheters illustrated in FIGS. 6A-7D. Another delivery catheter, configured to deliver the annuloplasty ring, may also form a part of the kit.

While in the foregoing specification the guard members, uses and kits of for an integrated structural guard member, configured to prevent leaflet(s) of the structural heart valve from extending cephalad to the annular plane a heart valve's disclosed, whether in conjunction with an annuloplasty ring assembly (or platform), have been described in relation to certain exemplary implementations, and many details are set forth for purpose of illustration, it will be apparent to those skilled in the art that the disclosure of the alignment methods, implementable using the systems disclosed herein are susceptible to additional implementations and that certain of the details described in this specification and as are more fully delineated in the following claims can be varied considerably without departing from the basic principles disclosed herein.

What is claimed:

1. A system for treating prolapse, flail, or billow of at least one leaflet of a native heart valve, the system comprising:
an annuloplasty ring configured for deployment using a delivery device, the annuloplasty ring comprising a plurality of anchors configured to be selectively deployed to secure the annuloplasty ring to a heart valve annulus of the native heart valve; and
a guard member comprising a plurality of struts coupled to a juncture at one end and coupled to the annuloplasty ring at another end, the guard member configured for deployment using the delivery device;
wherein a first strut and a second strut of the of plurality of struts together extend across the annuloplasty ring to prevent prolapse, flail, or billow of the at least one leaflet of the native heart valve.

2. The system of claim 1, further comprising a plurality of couplers, each coupler of the plurality of couplers coupled to a respective strut of the plurality of struts and configured to couple the respective strut to the annuloplasty ring.

3. The system of claim 2, wherein each coupler of the plurality of couplers is sized and configured to conform to a surface of the annuloplasty ring.

4. The system of claim 3, wherein the delivery device comprises a first delivery catheter configured to deliver the annuloplasty ring and a second delivery catheter configured to deliver the guard member.

5. The system of claim 1, wherein the juncture comprises a central strut.

6. The system of claim 1, wherein the juncture comprises an intersection forming a disc shape having a central aperture.

7. The system of claim 6, wherein the central aperture is configured to receive a coupling pin and the intersection is configured to engage the guard member with the delivery device via the coupling pin.

8. The system of claim 1, wherein the juncture comprises an intersection forming a disc shape.

9. The system of claim 1, wherein intersection is configured to be grasped by a plurality of graspers extending from the delivery device to engage the guard member with the delivery catheter.

10. The system of claim 1, wherein the guard member is configured to transition from a restrained position corresponding to a restrained shape to an unrestrained position corresponding to an unrestrained shape.

11. The system of claim 10, wherein each strut of the plurality of struts is configured to twist about a longitudinal axis of such strut.

12. A method for treating prolapse, flail, or billow of at least one leaflet of a native heart valve, the method comprising:
causing a delivery device to deliver an annuloplasty ring at the native heart valve, the annuloplasty ring comprising at least one anchor;
causing the annuloplasty ring to deploy the at least one anchor to secure the annuloplasty ring to a heart valve annulus of the native heart valve;
causing the delivery device to deliver a guard member to the native guard valve, the guard valve comprising a plurality of struts coupled to a juncture at one end; and
causing the delivery device to couple the guard valve to a surface of the annuloplasty ring,
wherein a first strut and a second strut of the of plurality of struts together extend across the annuloplasty ring to prevent prolapse, flail, or billow of the at least one leaflet of the native heart valve.

13. The method of claim 12, wherein the guard member comprises a plurality of couplers, each coupler of the plurality of couplers coupled to a respective one the plurality of struts, and wherein causing the delivery device to couple the guard member to a surface of the annuloplasty ring comprises causing the couplers to couple to the annuloplasty ring.

14. The method of claim 12, wherein the delivery device comprises a first delivery catheter configured to deliver the annuloplasty ring and a second delivery catheter configured to deliver the guard member.

15. The method of claim 12, wherein the juncture comprises an intersection forming a disc shape having a central aperture.

16. The method of claim 15, wherein the central aperture is configured to receive a coupling pin and the intersection is configured to engage the guard member with the delivery device via the coupling pin.

17. The method of claim 16, further comprising causing the coupling pin to disengage the intersection to release the guard member from the delivery device.

18. The method of claim 12, wherein the juncture comprises an intersection forming a disc shape and wherein the intersection is configured to be grasped by a plurality of graspers extending from the delivery device to engage the guard member with the delivery device.

19. The method of claim 18, further comprising causing the plurality of graspers to disengage the intersection to release the guard member from the delivery device.

20. The method of claim 12, further comprising causing, prior to causing the delivery device to couple the guard valve, the guard member to transition from a restrained position corresponding to a restrained shape to an unrestrained position corresponding to an unrestrained shape different from the restrained shape.
